(19) United States
(12) Patent Application Publication
(10) Pub. No.: US 2013/0196435 A1
(43) Pub. Date: Aug. 1, 2013

(54) STENCIL PATTERNING METHODS AND APPARATUS FOR GENERATING HIGHLY UNIFORM STEM CELL COLONIES

(71) Applicants: THE REGENTS OF THE UNIVERSITY CALIFORNIA, Oakland, CA (US); THE BOARD OF TRUSTEES OF THE LELAND STANFORD JUNIOR UNIVERSITY, Palo Alto, CA (US)

(72) Inventors: Luke P. Lee, Orinda, CA (US); Frank B. Myers, III, Berkeley, CA (US); Jason S. Silver, Piedmont, CA (US); Christopher K. Zarins, Menlo Park, CA (US); Oscar J. Abilez, Sunnyvale, CA (US)

(73) Assignee: THE REGENTS OF THE UNIVERSITY OF CALIFORNIA, Oakland, CA (US)

(21) Appl. No.: 13/738,210

(22) Filed: Jan. 10, 2013

Related U.S. Application Data
(60) Provisional application No. 61/585,097, filed on Jan. 10, 2012.

(51) Int. Cl. C12M 1/00; C12N 5/071; C12N 5/00

(52) U.S. Cl. CPC C12M 23/34; C12N 5/0068; C12N 5/0657
USPC 435/377; 435/395; 435/397; 435/305.1; 118/505

(57) ABSTRACT

A method for producing highly uniform cell colonies in a cell culture dish with the use of stencils made from an elastomeric sheet sized to fit within the cell culture dish, having a singular opening or a plurality of openings of a number, pitch and diameter configured to optimally control the geometric growth parameters of a cell colony. The uniform cell colonies are produced by placing the stencil in a cell culture dish and hydropilizing the stencil. The stencil is overlayed with cell culture media and seeded with seed cells that are preferably grown for at least a day before the stencil is removed to produce a pattern of seeded cells with controlled pitch, colony diameter and density within the culture dish that grow to become highly uniform cell colonies. A kit with culture dish, stencil, culture media and growth media is also provided.

### CROSS-REFERENCE TO RELATED APPLICATIONS

This application is a nonprovisional of U.S. provisional application Ser. No. 61/585,097 filed on Jan. 10, 2012, incorporated herein by reference in its entirety.

### STATEMENT REGARDING FEDERALLY SPONSORED RESEARCH OR DEVELOPMENT

This invention was made with Government support under Grant Number HR0011-06-1-0050 awarded by the Defense Advanced Research Projects Agency (DARPA). The Government has certain rights in the invention.

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to tissue culture devices and methods, and more particularly to a stencil patterning method for generating uniform cellular and tissue colonies allowing control over geometric growth parameters such as colony pitch, colony diameter in two or three dimensions and the number of colonies.

2. Background Discussion

Stem cells hold the promise of producing functional tissues that can replace those lost due to disease or injury. Stem cells exhibit “pluripotency”, meaning that they have the potential to become any cell type in the body. New organ tissues, such as those found in the heart, liver, or nervous system, can be created from stem cells through the process of “differentiation”. However, one major challenge in developing tissue replacement therapies is the heterogeneity and low yield associated with stem cell differentiation. It is well-established that mechanical factors associated with the cellular microenvironment, including cell shape and density, influence stem cell differentiation and cellular behaviors in general. Stem cells form isolated colonies in culture, and the geometry of these colonies can have a profound impact on their capacity for differentiation. Current commercialized technology for controlling the size and shape of embryoid body formation has demonstrated that the size of embyroid bodies is important for differentiation.

However, many existing differentiation techniques do not involve the formation of embryoid bodies, and instead induce differentiation from 2D monolayer cultures of stem cells. Current 2D stem cell culturing protocols lack control over colony geometry because they allow for random attachment to surrounding tissue-culturing surfaces. This leads to unpredictable stem cell growth, which ultimately hurts our ability to successfully control the cell fate and differentiation into specific cell types. Considering the importance of geometric factors in cell growth, there is a need for devices and methods that control growth to limit the variability of colony formation. The present invention satisfies this need, as well as others, and is generally an improvement in the art.

### BRIEF SUMMARY OF THE INVENTION

By patterning an extracellular matrix (ECM), such as Matrigel, colony formation, growth, and geometries can be highly regulated. The utility of controlling all of the geometric factors of stem cell colonies allows for the development of high yielding protocols for differentiation. By using stencils made of silicone or other suitable materials, a standard tissue culture plate can be converted into a cell patterning substrate while still using the normal ECM plating procedures. Stencil patterning may thus be useful as a means of standardizing and accelerating cell production for clinical tissue engineering. Additionally, as a research tool, controlling the geometry of cell colonies can help us recapitulate and better understand the stem cell niches that occur during development. Although a scheme adapted for stem cells is presented as an illustration, it will be understood that the methods can be adapted to colonization of other cell lines and cell types.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a flow diagram schematically illustrating a stencil patterning method for generating highly uniform stem cell colonies according to one embodiment of the invention.

FIG. 2 illustrates growth of stem cell colonies into a stencil.

FIG. 3 and FIG. 4 are a photograph and schematic illustration, respectively, of a stenciled pattern of cell colonies showing control over the geometric growth parameters.

FIG. 5 shows time-lapse microscopy results of plated patterns with different seeding densities over a six day growth period.

FIG. 6 through FIG. 8 are graphs of colony geometries with three different seeding densities and an unpatterned scrape passage for comparison.

FIG. 9 shows time-lapse image results over a four day period of stenciled colonies with a layer of Matrigel added after day two which allows the cells to proliferate beyond the initial patterns.

FIG. 10 shows a comparison of immunostaining results for patterned vs. unpatterned stem cells, along with a cartoon illustrating the positive effects that patterning has on cell signaling uniformity. The cells are stained for the pluripotency marker SSEA4.

FIG. 11 is a diagram of a pseudocolored heatmap showing that Nanog (pluripotency marker) is more uniformly expressed through the cross-section of a stem cell colony when the colony is patterned vs. when it is left unpatterned.

FIG. 12 shows a graph which compares expression of SSEA4 for patterned vs. unpatterned cells using flow cytometry, showing that patterning improves pluripotency.

FIG. 13 shows a graph which compares cardiomyocyte differentiation yield between patterned and unpatterned stem cells, showing that patterning improves yield by nearly 3×.

FIG. 14 is a photograph which illustrates that stem cell differentiation (cardiomyocyte differentiation, in this case) tends to follow patterned geometries, leading to broad applications in basic science and translational research.

### DETAILED DESCRIPTION OF THE INVENTION

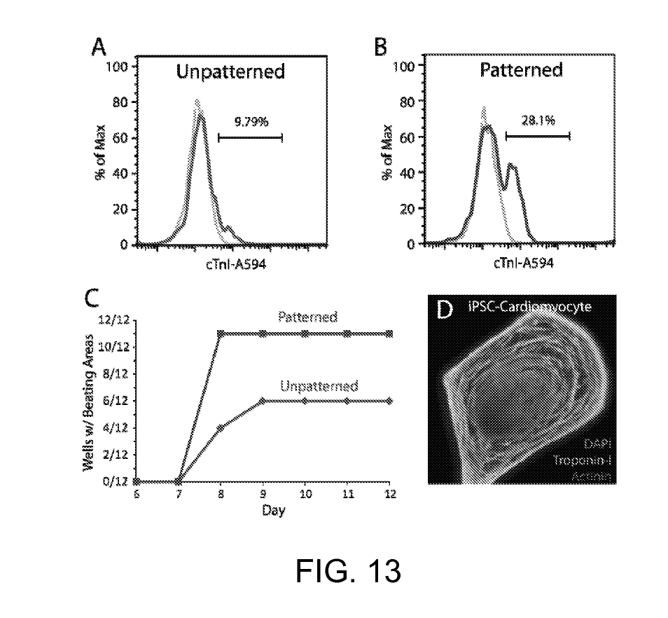

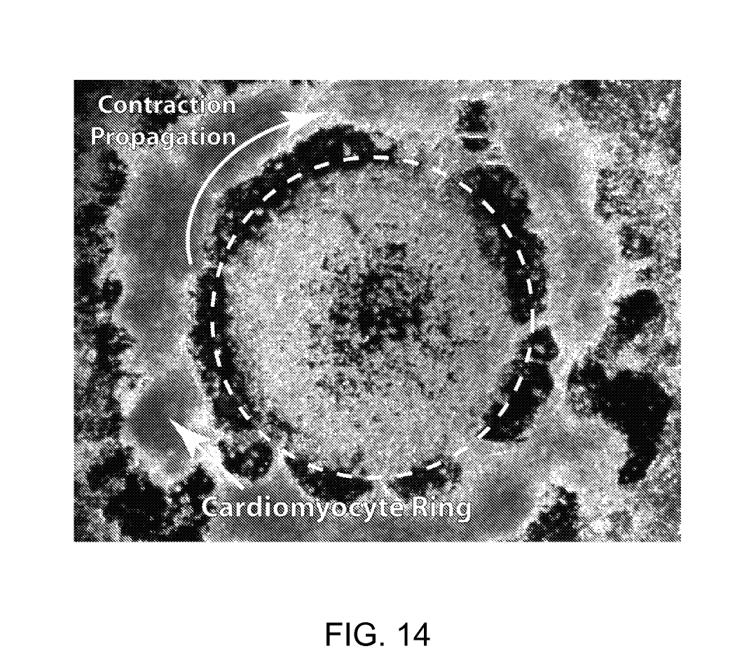

Geometric factors including the size, shape, and density of pluripotent stem cell colonies, as well as the spacing between neighboring colonies, play a significant role in the maintenance of pluripotency and in cell fate determination. These factors are impossible to control using standard tissue culture methods. As such, there can be substantial batch-to-batch variability in cell line maintenance and differentiation yield. We have developed a simple, robust technique for patterning Matrigel using a thin silicone stencil. It has been shown that patterned arrays of Matrigel spots lead to human induced pluripotent stem cell (hiPSC) colonies that are highly uniform in growth rate, size, and shape. Patterned cell colonies are capable of undergoing directed differentiation into spontaneously beating cardiomyocyte clusters, for example. When they do, we have observed that patterned cardiomyocyte differentiation leads to a higher yield (see FIG. 13) and a far more predictable geometry (see ring formation, FIG. 14). This has broad applications in regenerative medicine, tissue engineering, drug screening, and fundamental developmental biology. It is anticipated that this patterning method can improve both yield and repeatability in many stem cell differentiation procedures as well as with other cell lines.

The apparatus of the present invention can be used to standardize the growth of stem cells in vitro. Although we focus specifically on stem cell applications as an illustration, any cell type could be used with this invention. This technique offers greater control of colony geometry, size, spacing, and density, and allows for the optimization of differentiation conditions. When specifically considering stem cell culture, controlling the physical properties of colonies reduces the number of potential variables allowing for more precise experiments with consistent colony characteristics. This extracellular matrix (ECM) coating protocol can be implemented in all stem cell culture for not only the maintenance of cells, but also the differentiation of cells.

The best implementation of this invention would be to integrate this technique into current stem cell culturing and differentiation protocols.

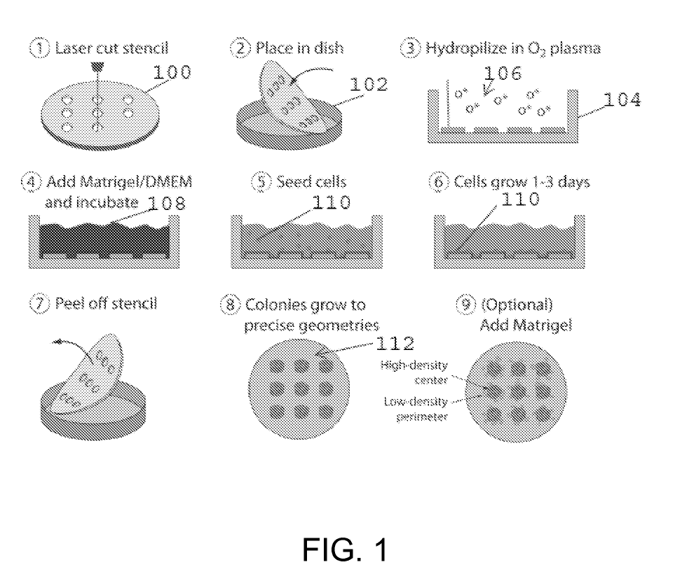

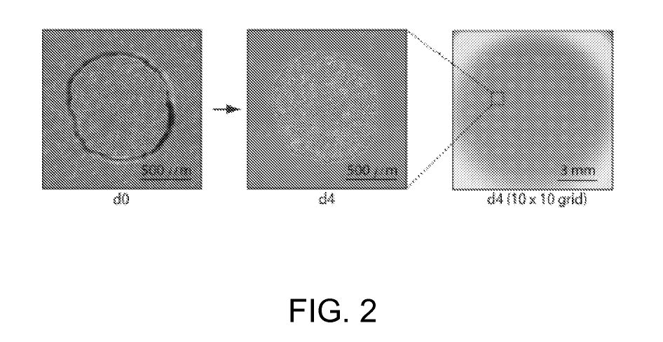

FIG. 1 illustrates the stencil patterning method according to one embodiment of the invention. At step 1, stencils 100 are cut from an elastomer sheet such as polydimethylsiloxane (PDMS) using a commercial CO₂ laser cutter/engraver or other methods such as waterjet cutting, soft lithographic, or mechanical patterning. The stencils are cut to fit a 35 mm round culture dish 102. Afterwards, at step 3, the stencils are cleaned and sterilized and placed into a standard polystyrene culture dish 104. The stencil attaches uniformly to the dish via van der Walls forces. At step 3, the stencil surface is hydrophilized with oxygen plasma to avoid bubbles at the stencil surface 106 (other methods may be used, such as ethanol treatment or vacuum treatment), and the ECM protein solution 108, Matrigel in this case, is added at step 4. Following Matrigel incubation, cells 110 are seeded onto the patterns and allowed to attach (step 5). The stencil may be removed immediately following attachment or the stencil may be left on for several days (step 6) and the stencil may then be removed (step 7). The colonies 112 grow to fill the pattern defined by the stencil with very high fidelity (step 8). Optionally, at step 9, Matrigel may be added back into the dish (by adding it to serum-free medium and allowing it to incubate briefly). Depending on the seeding density, cells will grow and fill the patterns in approximately 3-7 days as illustrated in FIG. 2.

In our pluripotency experiments, a 10×10 grid of 1 mm circles was used, and human induced pluripotent stem cells (hiPSCs) showed very good seeding uniformity across the entire pattern array. In our cardiomyocyte differentiation experiments, a single 2 mm colony was used, and it was observed that cardiomyocytes tended to arise along the perimeter of this colony, suggesting that the patterns can help guide developmental signaling and morphogenesis, in addition to providing better repeatability for these protocols.

While we have worked only with round colonies, any geometry could be used, and previous work in cell patterning has shown interesting geometry-dependent behavior.

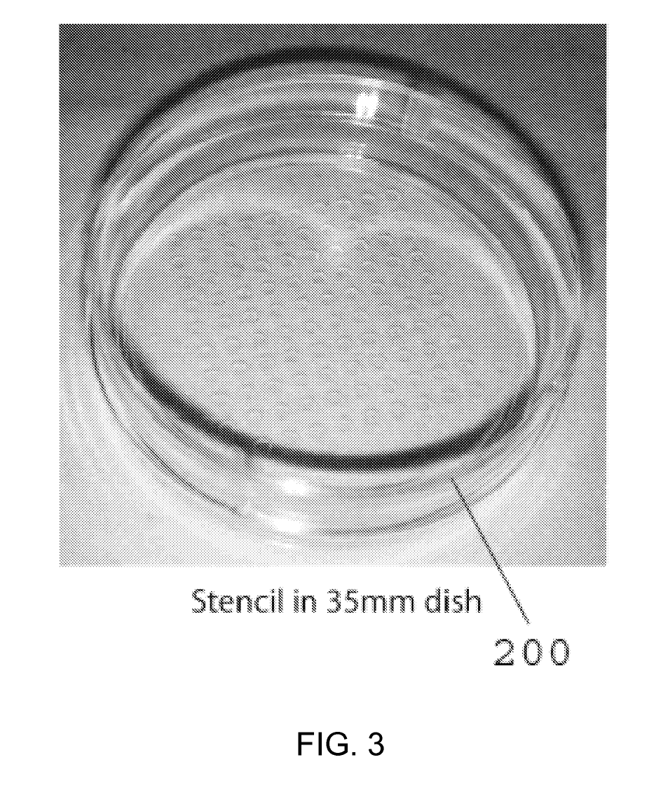

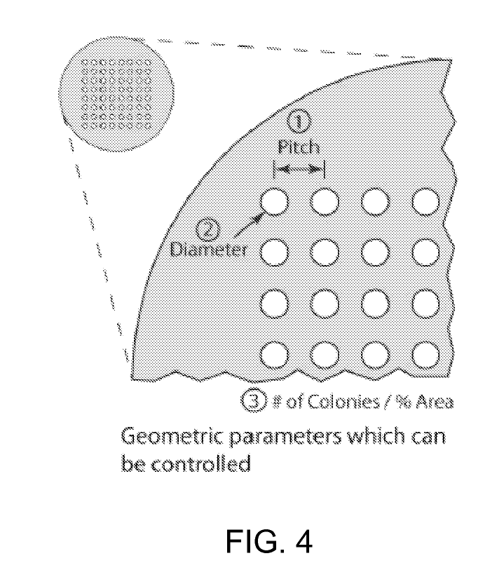

FIG. 3 and FIG. 4 illustrate parameters that can be controlled. FIG. 3 shows an example of a patterned 35 mm dish 200, with the stencil still adhered to the dish. In our experiments, we have worked exclusively with round colony patterns in grid arrays using round culture dishes, but this method is certainly not limited to this geometry. FIG. 4 illustrates several geometric parameters that can be tuned in the array: colony pitch 1, colony diameter 2, and the number of colonies 3 on the dish (or the overall area which is covered by cells). It is impossible to directly control these parameters with conventional culture techniques.

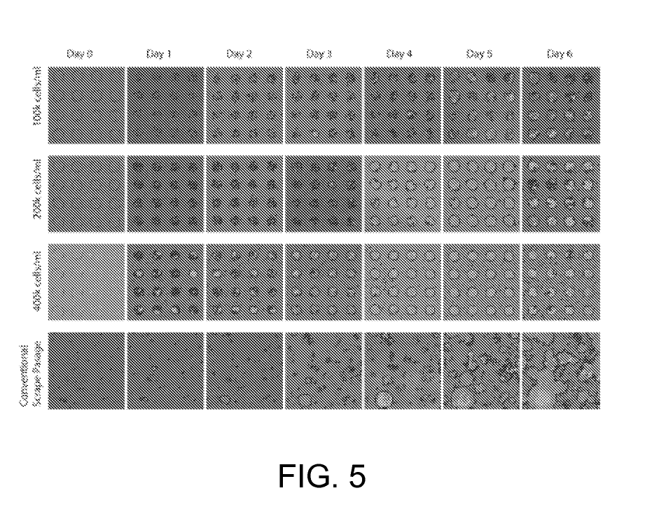

FIG. 5 shows time-lapse microscopy results of hiPSC culture at different seeding densities. Shown in FIG. 5 are phase-contrast microscopy mosaics of a section of a 10×10, 1 mm grid pattern of hiPSC cells at different initial seeding densities. Note that the lower densities take longer to fill the patterns, whereas the high concentration (400 k cells/ml) completely fills the patterns by day 4. The pattern outline indicates the outer perimeter of the colonies while the lighter spots that are present within the outline indicates vacancies within the colonies. Note the substantial heterogeneity in the unpatterned, conventional scrape passage culture.

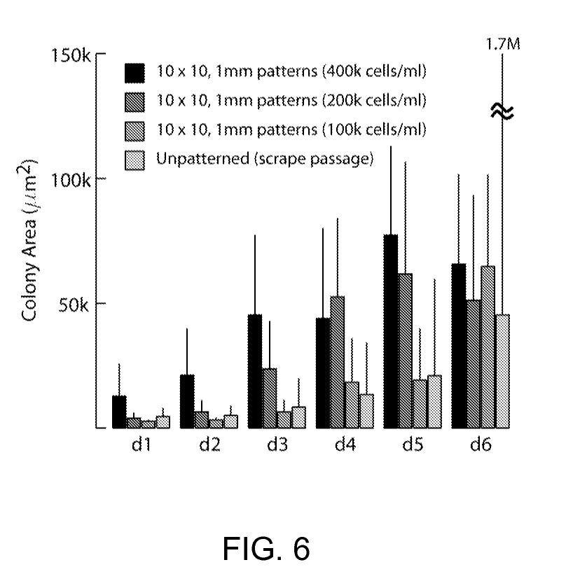

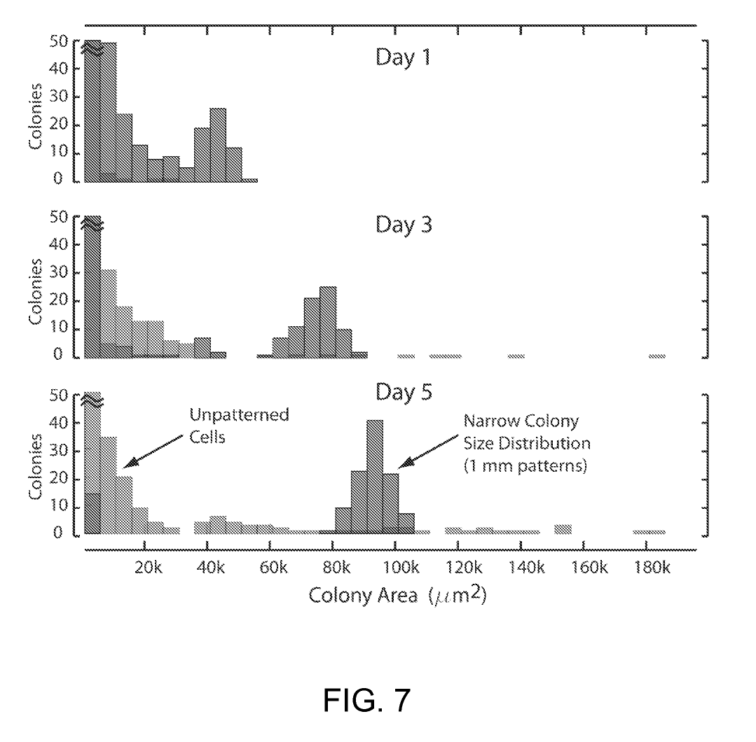

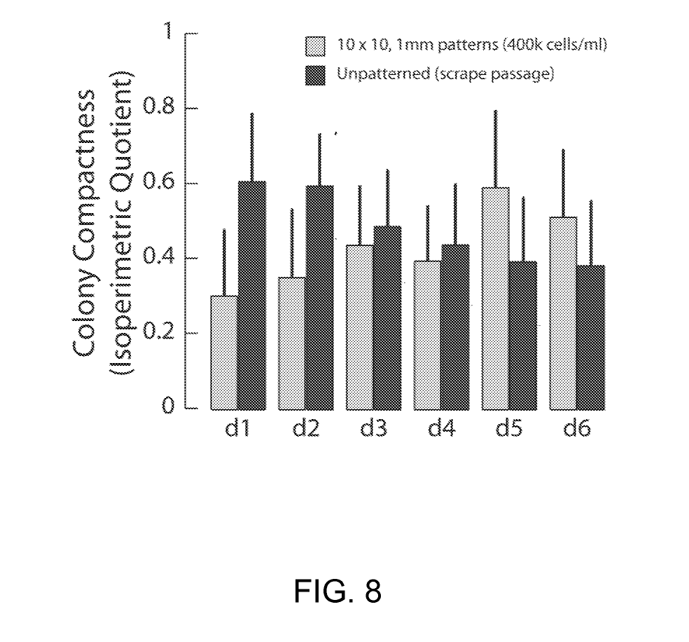

FIG. 6 through FIG. 8 illustrate quantitative analysis of the colony geometries. Referring to FIG. 6, colony area, as measured via phase-contrast microscopy and automated image analysis, shows a more controlled, linear growth in the patterned colonies versus a more heterogeneous (larger standard deviation), exponential growth in the unpatterned case. Patterned colonies tend to reach a maximum area and maintain that area for several days, whereas unpatterned colonies grow uncontrollably. FIG. 7 presents histograms showing the distribution of colony areas at different days for a patterned and unpatterned culture. Note that the patterned culture shows a substantially tighter distribution of colony sizes and a nearly linear growth rate. FIG. 8 illustrates colony compactness, as measured by the isoperimetric quotient. With this metric, a value of “1” indicates a perfect circle. Note that patterned cells tend towards circular colonies as they get larger whereas unpatterned cells tend away from circular colonies. Since circles are the simplest 2D geometry, differentiating cells from circular colonies may lead to more repeatable results.

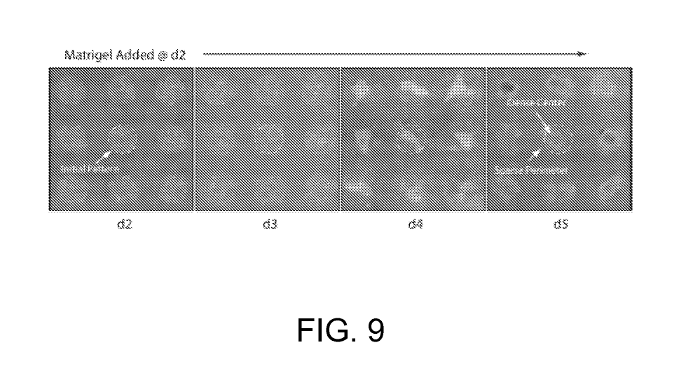

FIG. 9 illustrates cell growth when Matrigel is added after patterning to allow cells to proliferate beyond the patterns. Here, we show the results of adding Matrigel to the culture medium on day 2 (patterning/seeding=day 0). The colonies continue to proliferate beyond their initial boundaries, and interestingly, two different density zones emerge. It is thought that stem cells rely on density gradients to indicate germ layer fate, so this may be a way to control this parameter and allow colonies to grow beyond their initial boundaries during differentiation.

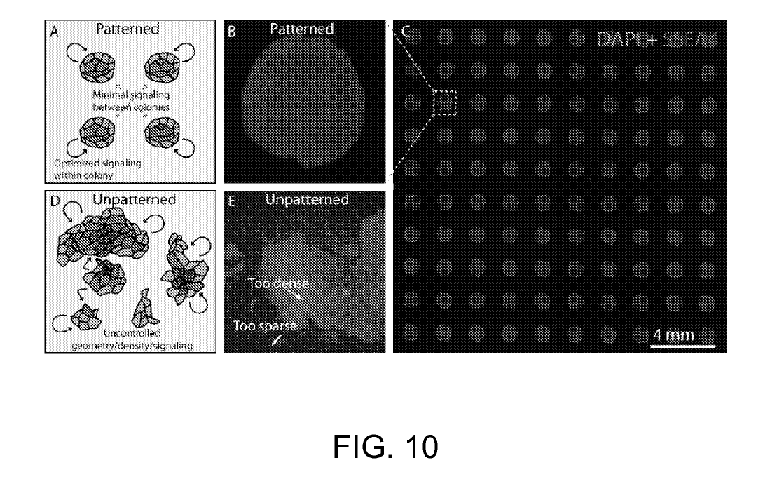

FIG. 10 Illustrates how cell colony geometry impacts paracrine and cell-contact signaling mechanisms, both of which are important for stem cell maintenance and differentiation. Panels A through C illustrate that geometric patterning can ensure each colony experiences similar stimulus conditions. A circular geometry ensures there is, at most, a radial dependence on these conditions. Panels D and E illustrate that unpatterned stem cell colonies show great heterogeneity as colonies grow asymmetrically and collide, making controlled experimentation and process scale-up difficult. As can be seen, stencil patterning leads to uniform pluripotency of iPSCs, as measured with SSEA4 expression, across all colonies within a culture. Using conventional clump passaging, cells which are very dense or very sparse tend to lose pluripotency.

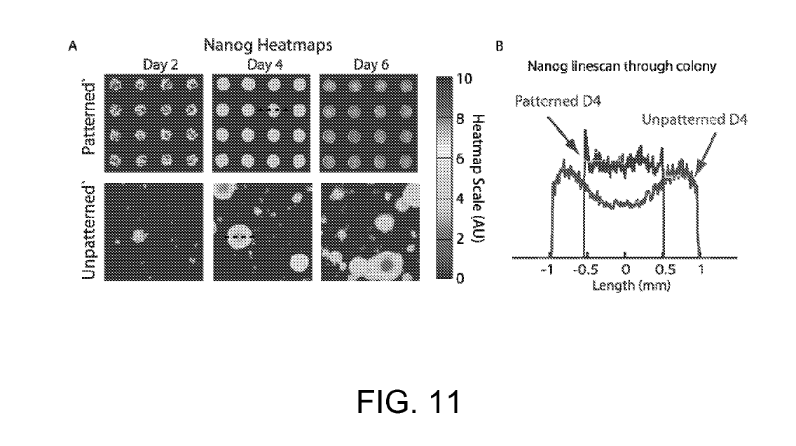

FIG. 11 shows that Nanog (pluripotency marker) is more uniformly expressed through the cross-section of a stem cell colony when the colony is patterned vs. when it is left unpatterned. Panel A presents pseudocolored heatmaps comparing Nanog expression in patterned and unpatterned colonies. Note the uniformity in patterned colonies and the nonuniformity in larger unpatterned colonies. This is further illustrated in the accompanying linescans, in Panel B where the black dotted lines in the heatmap images indicate the colonies from where the linescans are being drawn.

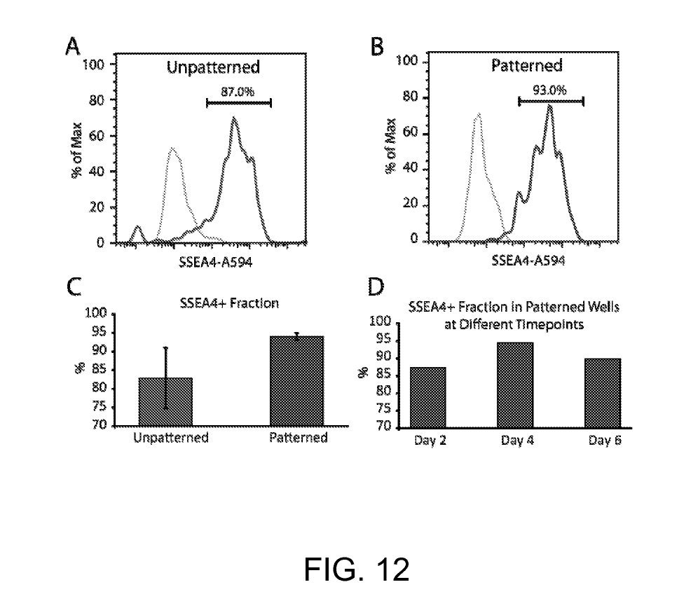

FIG. 12 compares expression of SSEA4 for patterned vs. unpatterned cells using flow cytometry. Panels A and B present flow cytometry data from cells at day 4 indicating that SSEA4 expression is more uniform in patterned colonies, which is consistent with our microscopy data. Panel C shows that a higher fraction of cells in patterned colonies are SSEA4+, and batch-to-batch variability is reduced (error bars signify standard deviation, n=3). Panel D shows that, consistent with our microscopy data, SSEA4 expression is slightly higher at day 4 than at day 2 or day 6. A survey of pluripotency and early germ layer differentiation markers revealed that bulk expression is equivalent between patterned and unpatterned colonies (error bars signify standard deviation, n=3), as is the batch-to-batch variability.

FIG. 13 compares cardiomyocyte differentiation yield between patterned and unpatterned stem cells, showing that patterning improves yield by nearly 3×. Panels A and B show cardiomyocyte differentiation yield and robustness is improved with stencil patterning. Cells were immunostained for cardiac troponin-I (cTnI) and analyzed by flow cytometry. Panel C illustrates that patterned wells show a nearly 3-fold increase in the cTnI+fraction. Spontaneous beating of cardiomyocyte colonies occurred in 11/12 wells on the same day (day 8) in patterned wells, whereas unpatterned wells showed greater heterogeneity—some wells initiate beating on day 8 while others start at day 9, and only 6/12 wells successfully differentiate to beating cardiomyocytes. Panel D illustrates that differentiated cardiomyocytes display characteristic sarcomeric banding with troponin-I (cTnI) and actinin.

FIG. 14 illustrates the results of cardiomyocyte differentiation from circular 2 mm colonies. The dashed line indicates the original patterned colony boundary. Just outside of this boundary is where cardiomyocytes typically arise, leading to a 3D ring geometry. As a result of this geometry, electrophysiological signal propagation tends to occur in a ring shape as well. Such control over stem cell fate and geometric patterning of differentiated tissues has broad applications in drug screening, tissue engineering, and developmental biology.

Table 1 compares the invention with other ECM patterning techniques. It will be appreciated that our inventive stencil patterning apparatus and method offers many advantages over other ECM patterning techniques that have been developed recently. Stem cell biology is a rapidly growing field with a number of companies producing tools specifically for the stem cell community. A simple cell patterning method such as this could be readily adapted to existing tissue culture product lines and could be adapted for a variety of experimental applications where cell culture geometry is deemed relevant. Tissue culture dishes could be sold pre-patterned, or the stencils themselves could be sold separately as well.

There are several advantages to the present invention versus normal tissue culture plates and alternative methods to patterning ECM. Other ECM patterning techniques include photolithography, soft lithography, Robotic DNA spotters and direct write systems, inkjet printing, and chemically-switchable surfaces. By using a PDMS (polydimethylsiloxane) elastomeric stencil for ECM patterning, conventional ECM plating protocols can be easily adapted to this patterning technique. Stencil patterning has been demonstrated before, but fabrication techniques either involved the use of a photosensitive polymer or spin casting prepolymer and allowing it to cure on a mold. Elastomeric stencils that are laser or die cut from mass produced elastomeric sheets would seem to be the most promising form from a manufacturability standpoint. Other techniques subject ECM or cell suspensions to high temperatures, pressures, or shear forces. Additionally, Matrigel is unique in that it must be applied cold and allowed to gel over time. The thickness of the resulting gel layer can be tuned by varying the Matrigel concentration. These parameters are difficult to control with other patterning techniques. Additionally, because the stencils are preferably with a robotic laser cutter, stencils can be easily mass-produced for rapid prototyping. Alternative embodiments may use different materials or different manufacturing methods. The essential component is a self-adhesive stencil that can be easily applied and removed (by hand or robotically) to the bottom of common tissue culture slides, plates, or dishes. Additionally, the stencil may include wells or indentations around each opening so that droplets of ECM/cells/media can be applied to the opening rather than flooding the entire plate with liquid. The wells/indentations would help confine the droplets. The use of droplets reduces reagent/cell consumption.

It has also been shown that the pluripotency, growth rate, and differentiation capacity of patterned stem cells are comparable to their unpatterned counterparts.

Because we use laser ablative cutting to produce the stencils, our resolution is generally limited to >200 μm. But for the proposed application, this resolution is more than adequate, as stem cell colonies generally grow to 1-3 mm in diameter. Other manufacturing techniques such as injection molding or spin casting on with molds or mechanical stamping could be employed to produce stencils with single cell resolution.

From the discussion above it will be appreciated that the invention can be embodied in various ways, including the following:

1. A method for producing highly uniform cell colonies, comprising: placing a stencil in a cell culture dish patterned with a singular opening or a plurality of openings of a selected size, spacing and density; hydropilizing the stencil; overlaying the stencil with cell culture media; seeding the cell culture media with seed cells; growing the seed cells in the cell culture media; and removing the stencil to produce a pattern of seeded cells with controlled pitch, colony diameter and density within the culture dish; wherein controlled pitch, colony diameter and density within the culture dish produces highly uniform cell colonies.

2. The method of embodiment 1, wherein the stencils are elastomeric stencils cut from a PDMS (polydimethylsiloxane) material.

3. The method of embodiment 1, wherein the stencils have circular openings with a diameter ranging from approximately 200 μm to approximately 3 mm.

4. The method of embodiment 1, wherein the stencils have a distance between openings ranging from approximately 500 μm to approximately 3 mm.

5. The method of embodiment 1, further comprising depositing droplets of extracellular matrix (ECM), cells or growth media in a well surrounding each opening in the stencil; wherein growth media, cells or extracellular matrix (ECM) can be applied to individual openings rather than the entire surface of the stencil.

6. The method of embodiment 1, wherein the cell culture media is an extracellular matrix (ECM) gel.

7. The method of embodiment 1, wherein the stencil/culture dish is hydrophilized prior to adding culture media using oxygen plasma.

8. The method of embodiment 1, wherein the stencil/culture dish is hydrophilized prior to adding culture media using a wetting solvent such as ethanol

9. The method of embodiment 1, wherein the stencil/culture dish is vacuum treated prior to adding culture media to avoid bubble formation.

10. The method of embodiment 6, wherein the extracellular matrix (ECM) is Matrigel.

11. The method of embodiment 10, further comprising optimizing the viscosity of the cell culture media by varying the concentration of Matrigel.

12. The method of embodiment 1, wherein the seed cells are seeded with a seeding density of between approximately 100 k cells/ml and approximately 400 k cells/ml.

13. The method of embodiment 1, further comprising applying a second layer of cell culture media over the unpatterned areas of plate after the stencil is removed; wherein the cell colonies can grow beyond their initial borders.

14. The method of embodiment 1, wherein an extracellular matrix (ECM) and a cell suspension are added simultaneously.

15. A method as recited in embodiment 1, wherein the cell colonies are differentiated into cardiomyocytes.

16. A method as recited in embodiment 1, wherein the patterns of seeded cells are round and lead to ring-shaped differentiated cell cluster geometries.

17. A method as recited in embodiment 15, wherein the cardiomyocyte cells lead to predictable electrophysiological propagation, permitting their robust alignment to electrodes or some other recording apparatus.

18. A stencil for producing highly uniform cell colonies, comprising an elastomeric sheet sized to fit within a cell culture dish, said sheet having a singular opening or a plurality of openings of a number, pitch and diameter configured to optimally control the geometric growth parameters of a cell colony.

19. The stencil of embodiment 15, wherein the elastomeric sheet is made from a PDMS (polydimethylsiloxane) material.

20. The stencil of embodiment 15, wherein the stencil has circular openings with a diameter ranging from approximately 200 μm to approximately 3 mm.

21. The stencil of embodiment 15, wherein a distance between the openings ranges from approximately 500 μm to approximately 3 mm.

22. The stencil of embodiment 15, further comprising a plurality of indentations fluidly coupled to the openings configured to receive droplets of extracellular matrix (ECM), cells or growth media and dispense the droplets to the stencil opening.

23. A kit for producing highly uniform cell colonies, comprising: at least one tissue culture dish; at least one stencil sized to fit within the tissue culture dish, said stencil having a singular opening or a plurality of openings of a number, pitch and diameter configured to optimally control the geometric growth parameters of a cell colony; a hydropilizer; cellular culture media; and cellular growth media.

24. The kit of embodiment 20, wherein the stencils are elastomeric sheets made from a PDMS (polydimethylsiloxane) material.

25. The kit of embodiment 20, wherein the stencils have circular openings with a diameter ranging from approximately 200 μm to approximately 3 mm.

26. The kit of embodiment 20, wherein the stencils have a distance between openings ranging from approximately 500 μm to approximately 3 mm.

27. The kit of embodiment 20, wherein the cell culture media is an extracellular matrix (ECM) gel.

28. The kit of embodiment 24, wherein the extracellular matrix (ECM) is Matrigel.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
Comparison With Other ECM Patterning Techniques

| | | | Inkjet | | Robotic DNA | |
|---|---|---|---|---|---|---|
| | Soft lithography | Photolithography | Printing | Switchable Surfaces | Spotter | Stencil Patterning |
| Compatible | No (requires drying | No (use silane | No (heats | No (requires a linker | No | Yes (same protocol) |
| w/ Standard | on pdms) | chemistry to attach | matrigel) | molecule) | | |
| Matrigel | | ECM) | | | | |
| Protocol | | | | | | |
| Rapid | No | No | Yes | No | Yes | Yes |
| Prototyping | | | | | | |
| Cost of | High | High | Low | High | High (equipment | Low (laser cutter or |
| Equipment | | | | | cost) | blade cutter) |
| Time to make | Hours | Hours | Minutes | Hours | Minutes | Minutes |
| patterns | | | | | | |
| Resolution | >2 μm | >10 nm | >350 μm | >1 μm | >100 μm | >500 μm |
| Compatible | No | No | Yes | No | No | Yes |
| with 3D ECM | | | | | | |

What is claimed is:

1. A method for producing highly uniform cell colonies, comprising:
placing a stencil in a cell culture dish patterned with a singular opening or a plurality of openings of a selected size, spacing and density;
hydropilizing the stencil;
overlaying the stencil with cell culture media;
seeding the cell culture media with seed cells;
growing the seed cells in the cell culture media; and
removing the stencil to produce a pattern of seeded cells with controlled pitch, colony diameter and density within the culture dish;
wherein controlled pitch, colony diameter and density within the culture dish produces highly uniform cell colonies or improves the yield and/or geometric repeatability of stem cell differentiation.

2. A method as recited in claim 1, wherein the stencils are elastomeric stencils cut from a PDMS (polydimethylsiloxane) material.

3. A method as recited in claim 1, wherein said stencils have circular openings with a diameter ranging from approximately 200 μm to approximately 3 mm.

4. A method as recited in claim 1, wherein said stencils have a distance between openings ranging from approximately 500 μm to approximately 3 mm.

5. A method as recited in claim 1, further comprising:
depositing droplets of extracellular matrix (ECM), cells or growth media in a well surrounding each opening in the stencil;
wherein growth media, cells or extracellular matrix (ECM) can be applied to individual openings rather than the entire surface of the stencil.

6. A method as recited in claim 1, wherein the cell culture media is an extracellular matrix (ECM) gel.

7. A method as recited in claim 1, wherein the stencil/culture dish is hydrophilized prior to adding culture media using oxygen plasma.

8. A method as recited in claim 1, wherein the stencil/culture dish is hydrophilized prior to adding culture media using a wetting solvent such as ethanol

9. A method as recited in claim 1, wherein the stencil/culture dish is vacuum treated prior to adding culture media to avoid bubble formation.

10. A method as recited in claim 6, wherein the extracellular matrix (ECM) is Matrigel.

11. A method as recited in claim 10, further comprising:
optimizing the viscosity of the cell culture media by varying the concentration of Matrigel.

12. A method as recited in claim 1, wherein the seed cells are seeded with a seeding density of between approximately 100 k cells/ml and approximately 400 k cells/ml.

13. A method as recited in claim 1, further comprising:
applying a second layer of cell culture media over on the unpatterned areas of the plate after the stencil is removed;
wherein the cell colonies can grow beyond their initial borders.

14. A method as recited in claim 1, wherein an extracellular matrix (ECM) and a cell suspension are added simultaneously.

15. A method as recited in claim 1, wherein the cell colonies are differentiated into cardiomyocytes.

16. A method as recited in claim 1, wherein the patterns of seeded cells are round and lead to ring-shaped differentiated cell cluster geometries.

17. A method as recited in claim 15, wherein the cardiomyocyte cells lead to predictable electrophysiological propagation, permitting their robust alignment to electrodes or some other recording apparatus.

18. A stencil for producing highly uniform cell colonies, comprising:
an elastomeric sheet sized to fit within a cell culture dish, said sheet having a singular opening or a plurality of openings of a number, pitch and diameter configured to optimally control the geometric growth parameters of a cell colony.

19. A stencil as recited in claim 18, wherein the elastomeric sheet is made from a PDMS (polydimethylsiloxane) material.

20. A stencil as recited in claim 18, wherein said stencil has circular openings with a diameter ranging from approximately 200 μm to approximately 3 mm.

21. A stencil as recited in claim 18, wherein a distance between said openings ranges from approximately 500 μm to approximately 3 mm.

22. A stencil as recited in claim 18, further comprising:
a plurality of indentations fluidly coupled to said openings configured to receive droplets of extracellular matrix (ECM), cells or growth media and dispense the droplets to the stencil opening.

23. A kit for producing highly uniform cell colonies, comprising:
at least one tissue culture dish;
at least one stencil sized to fit within the tissue culture dish, said stencil having a singular opening or a plurality of openings of a number, pitch and diameter configured to optimally control the geometric growth parameters of a cell colony;
a hydropilizer;
cellular culture media; and
cellular growth media.

24. A kit as recited in claim 23, wherein the stencils are elastomeric sheets made from a PDMS (polydimethylsiloxane) material.

25. A kit as recited in claim 23, wherein said stencils have circular openings with a diameter ranging from approximately 200 μm to approximately 3 mm.

26. A kit as recited in claim 23, wherein said stencils have a distance between openings ranging from approximately 500 μm to approximately 3 mm.

27. A kit as recited in claim 23, wherein the cell culture media is an extracellular matrix (ECM) gel.

28. A kit as recited in claim 27, wherein the extracellular matrix (ECM) is Matrigel.
